(19) United States
(12) Patent Application Publication
(10) Pub. No.: US 2019/0117144 A1
(43) Pub. Date: Apr. 25, 2019

(54) HEART RATE VARIABILITY AND DROWSINESS DETECTION

(71) Applicants: Bruno D. Carraro, Royal oak, MI (US); Eric Kozlowski, Oakland Township, MI (US); Dino S. Nardicchio, Blooimfield, MI (US); Gary Benninger, West Bloomfield, MI (US); MAGNA SEATING INC., Aurora (CA)

(72) Inventors: Bruno D. CARRARO, Royal Oak, MI (US); Eric KOZLOWSKI, Oakland Township, MI (US); Dino S. NARDICCHIO, Bloomfield, MI (US); Gary BENNINGER, West Bloomfield, MI (US)

(73) Assignee: Magna Seating Inc., Aurora, ON (CA)

(21) Appl. No.: 16/091,644

(22) PCT Filed: Apr. 10, 2017
(86) PCT No.: PCT/US2017/026787

§ 371 Date: Oct. 5, 2018

Related U.S. Application Data
(60) Provisional application No. 62/319,854, filed on Apr. 8, 2016.

(51) Int. Cl. A61B 5/18; A61B 5/00; A61B 5/024; G08B 21/06

(52) U.S. Cl. CPC A61B 5/18; A61B 5/6893; A61B 5/7253; G08B 21/06; A61B 5/74; A61B 5/02405

(57) ABSTRACT

Methods, devices and systems are provided for detecting drowsiness or the alertness level of a driver of vehicle. A driver's heart rate variability level is determined based on signals received from one or more sensors placed in the vehicle, such as biometric sensors located in a seat assembly. In response to a low heartbeat variability indicating a drowsy or non-alert driver, one or more output signals may be generated to warn or notify the driver.

### CROSS-REFERENCE TO RELATED APPLICATIONS

This application claims priority to U.S. Provisional Application No. 62/319,854, filed on Apr. 8, 2016.

### TECHNICAL FIELD

Example embodiments relate to a seat assembly in a vehicle as well as systems and methods for detecting drowsiness of a driver and for alerting a driver.

### BACKGROUND

Numerous automotive accidents are caused by drowsy, sleeping or hypo-vigilant drivers. Others have attempted to address this issue and prevent accidents by creating technology to detect a lane departure of a vehicle, such as U.S. Pat. No. 8,354,932 to Schmitz. Other systems rely on the use of an electrooculogram (EOG) and image capture devices to monitor and interpret blinking patterns of the driver in order to detect drowsiness. See, for example, U.S. Pat. No. 8,306,271 to Yoda et al.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a block diagram illustrating a system in accordance with one embodiment of the present disclosure;

FIG. 2 is a schematic perspective view of an automotive vehicle interior;

FIG. 3 is a perspective view of a dashboard display of the automotive vehicle;

FIG. 4 is a close up view of a seat assembly for an automotive vehicle;

FIG. 5 is a side view of the automotive vehicle interior and a driver;

FIG. 6 is a flow chart of a method of detecting a low alertness level of a driver;

FIGS. 7, 8 and 9 are graphs of received sensor signals and R-R intervals;

FIG. 10 is a graph of R-R interval values over three time intervals;

FIG. 11 is a graph with a histogram representation of some of normalized data from FIG. 10; and

FIGS. 12, 13 and 14 are graphs of power spectral analysis determined for received sensor signals.

Like reference numerals are used throughout the Figures to denote similar elements and features.

### DETAILED DESCRIPTION OF EXAMPLE EMBODIMENTS

Many prior systems for detecting drowsiness of a driver are primarily reactive in that they detect or respond to the driver being drowsy, or to the operation of the vehicle by a sleeping or drowsy driver. The present application describes methods, devices and systems for detecting drowsiness or the alertness level of a driver of vehicle, preferably before the driver's state affects the operation of the vehicle. Embodiments described herein measure the driver's heart rate variability (HRV) through biometric sensors in the vehicle and a drowsy state is predicted by correlating HRV values to activation levels of the driver's central nervous system. Although illustrated in the present application with the example of an automotive vehicle, the described embodiments may apply to other vehicles operated by a seated driver.

Through the central nervous system, the brain regulates two motor systems, the voluntary motor system which provides for muscular control of the limbs, body and head; and the involuntary motor system, also known as the autonomic nervous system (ANS), which regulates internal organs like the heart, digestive system, lungs, bladder and blood vessels. The ANS is divided into 2 opposing sections: the parasympathetic nervous system which is responsible for “rest and digest” functions; and the sympathetic nervous system which is responsible for “fight or flight” functions. The interaction between the parasympathetic and sympathetic nervous systems is known as sympathovagal balance. The sympathovagal balance leads to variations in cardiac output, heart rate, blood flow, pupil dilation and digestive system.

Human heart rates are often calculated by measuring the number of contractions or beats per minute (bpm). A typical, healthy human is likely to have a heart rate (HR) of about 50 to 90 bpm. Heart rate variability (HRV) refers to the phenomenon of the variation in the time intervals between heartbeats. HRV rises as the dominance of the sympathetic system gains relevance over parasympathetic system, such as when a person is more alert and active. HRV falls when the parasympathetic system gains relevance, such as when a person is getting drowsy. Thus, the variation of HRV over time can provide a physiological indication of a person transitioning from an alert to a drowsy state.

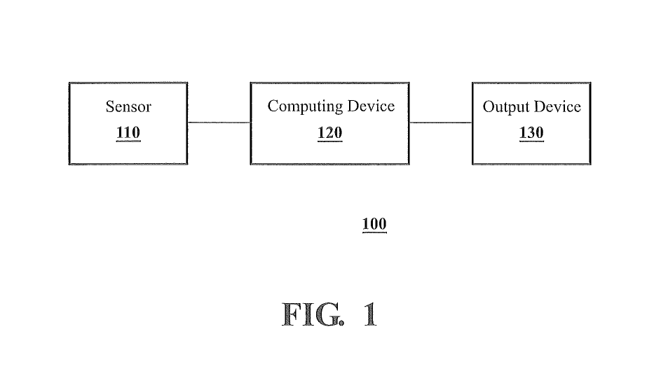

A block diagram of a system 100 for detecting drowsiness of a driver of a vehicle is illustrated in FIG. 1. The system 100 includes one or more sensors 110, a computing device 120, and one or more output devices 130. As described further below, the computing device 120 is configured to detect drowsiness or low alertness of the driver based on analysis of the signals received from the one or more sensors 110 and a determination of the HRV of the driver. If a drowsy state is predicted, the computing device 120 sends signals to activate one or more output devices 130 in order to notify the driver. In some embodiments, the system 100 includes a seat assembly of the vehicle, a user interface of the vehicle such as a dashboard display, or both the seat assembly and the user interface.

The computing device 120 has a processor and a memory which is configured to store and execute instructions for methods for detecting drowsiness as described herein. The computing device 120 may be a separate module or component, such as a programmable chip or application specific integrated circuit, or it may be part of the another computing device present in the vehicle. In some embodiments, the computing device 120 may be configured to support wired and/or wireless communications with other systems of the vehicle and with the sensor 110 and output device 130. The computing device 120 may include a user interface and the computing device 120 may be configured to support a user programming or configuring the computing device 120 and/or a user obtaining data or reports of information stored by the computing device 120 through the user interface. The computing device 120 may include additional signal processing circuitry or functionality in order to filter signals received from the sensor 110.

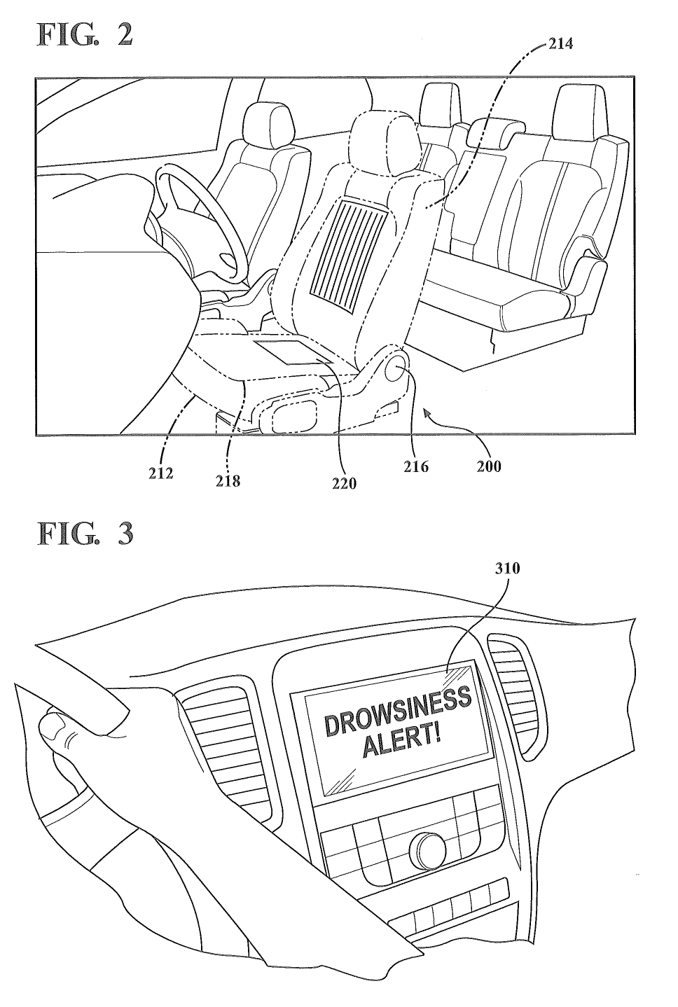

FIG. 2 illustrates an example view of an automotive vehicle interior and the environment for the possible placement of sensors 110 and output devices 130. The vehicle includes a seat assembly 200 which includes a generally horizontal seat cushion 212 and a generally upright seat back 214 for supporting a seat occupant within the vehicle. The seat back 214 is typically operatively coupled to the seat cushion 212 by a recliner assembly 216 for providing pivotal movement between an upright seating position and a plurality of reclined seating positions. The seat occupant is referred to herein as the driver.

Each of the seat cushion 212 and seat back 214 commonly includes a molded resilient cellular foam pad (not shown) encased in a trim cover assembly 218, which may be of cloth, vinyl, or leather. The one or more sensors 110 may be placed in the seat assembly 200 behind the trim cover assembly 218, either behind/below or in front/on top of the foam pad. In some embodiments, the one or more sensors 110 may be integrated into a layer of the trim cover assembly 218. In some embodiments, the one or more sensors 110 may be provided as part of a heating and cooling system for the seat assembly 200. In some embodiments, the one or more sensors output devices 130 may be provided as part of the heating and cooling system for the seat assembly 200.

Each sensor 110 comprises a biometric sensor which is mounted in the vehicle in order to gather data about the driver. Multiple sensors 110 of the same type may be used, or the sensors 110 may comprise multiple sensors of different types. Example types of sensors include a capacitive sensor, a radio frequency (RF) sensor, an impedance sensor, a permittivity sensor, or an ultrasensitive pressure transducer. Each type sensor is described below.

Capacitive sensors monitor a voltage change over time caused by the polarization and depolarization of the heart muscle when beating. Electrodes on the sensor interact with the body of the driver to determine those variations. In one embodiment, capacitive sensors are located in or on the seat back 214.

Radio Frequency (RF) sensors are composed of a transceiver and an antenna. The transceiver modulates and issues a radio frequency signal through an antenna to the driver's body. The mechanical and electromagnetic changes in the body caused by the driver's heartbeat and respiration modulate the signals that bounce back to the antenna. This modified signal is received or captured by the RF sensor. In one embodiment, RF sensors are located in or on the seat back 214, and/or in or on the seat cushion 212.

Impedance sensors monitor the bio-impedance signal from the driver's body obtained due to blood volume changes and blood resistivity changes between heart beats. In one embodiment, impedance sensors are located in or on the seat back 214.

Permittivity sensors create an electromagnetic field above a surface of the sensor. During polarization and depolarization of the driver's heart muscle, the electromagnetic field is affected and the disturbances in the field are recorded by the permittivity sensor. In one embodiment, permittivity sensors are located in or on the seat back 214.

Ultrasensitive pressure transducers operate on the principle that every time the heart beats, a mechanical pulse is generated throughout the driver's body. This mechanic pulse creates a signal also known as a ballistocardiogram. Ultrasensitive pressure transducers are made of piezo-resistive and piezo-electric materials that sense the fluctuation of these ballistic forces over time. Ultrasensitive pressure transducers can be located in or on the seat back 214, and/or in or on the seat cushion 212.

In some embodiments, the seat assembly 200 includes an electrostatic discharge (ESD) mat 220. The ESD mat 220 is an anti-static device that helps eliminate static electricity by having a controlled low resistance. The mat is grounded to the vehicle in order to discharge the static electricity at a slow rate. In one embodiment, the ESD mat 220 is located within the seat cushion 212 as shown in FIG. 2.

FIGS. 3, 4 and 5 illustrate additional example views of the automotive vehicle interior and the environment for the possible placement of sensors 110 and output devices 130. An output device 130 is a device capable of generating output signals, such as alarms or notifications, to alert a driver to a potential state of drowsiness or to an actual state of the driver being drowsy or asleep. In some embodiments, the one or more output devices 130 may be integrated into a layer of the trim cover assembly 218. In some embodiments, the one or more output devices 130 may be provided as part of a heating and cooling system for the seat assembly 200.

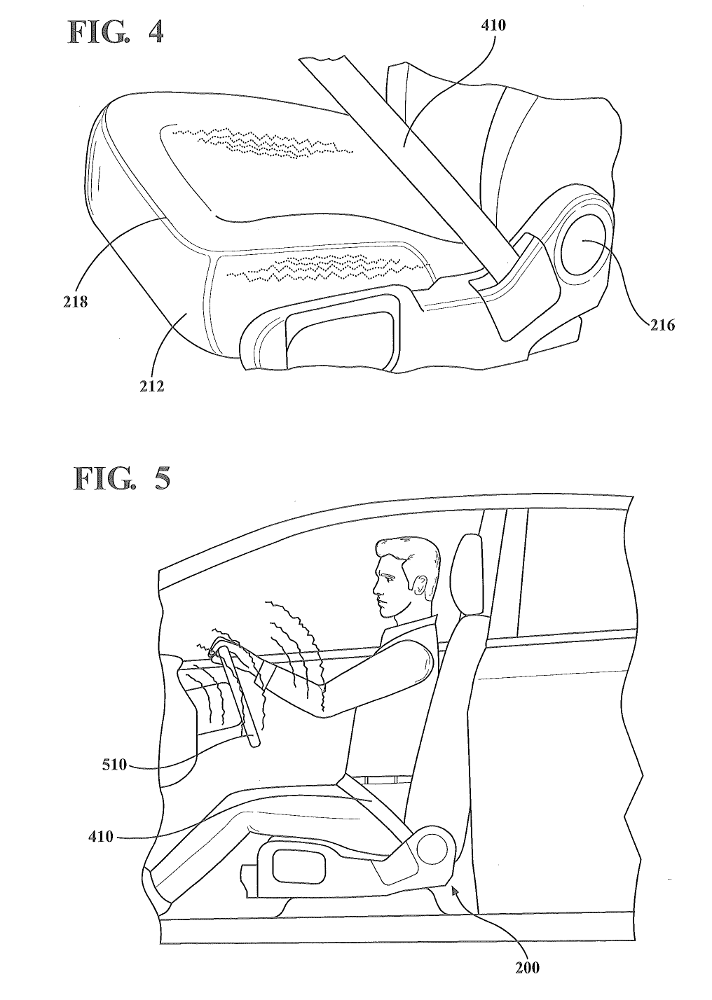

As shown in FIG. 3, an output device 130 may include a dashboard indicator or infotainment system generating an audio, visual, or audio/visual notification such as an alarm and or message 310. FIG. 4 illustrates a close-up view of the seat assembly 200. An output device 130 may provide a haptic alert such as a vibration which may be felt by the driver. The haptic alert may be provided via a vibration or other sensory disturbance of the seat cushion 212, the seat back 214, the restraint system 410, and/or the steering wheel 510 as illustrated in FIG. 5, or any combination thereof.

Multiple output devices 130 may be activated by the computing device 120 at or around the same time in order to generate multiple audio, visual, audio/visual, and/or haptic signals to alert the driver to a state in which the driver may not have a sufficient level of alertness to safely operate the vehicle. The output devices 130 may be activated if the driver's measured HRV predicts a low alertness level. In one embodiment, the output devices 130 are deactivated once the driver's measured HRV increases and the level of predicted alertness is sufficient for operation of the vehicle. In some embodiments, the output devices 130 may be deactivated based on other feedback signals, such as the receipt of an input through a user interface of the system 100 or through an input of the infotainment system, to acknowledge the alarm or warning signal. Other feedback may include the slowing or stopping of the vehicle. These other feedbacks or inputs may be used alone or in combination with the measured HRV of the driver to deactivate the output device 130.

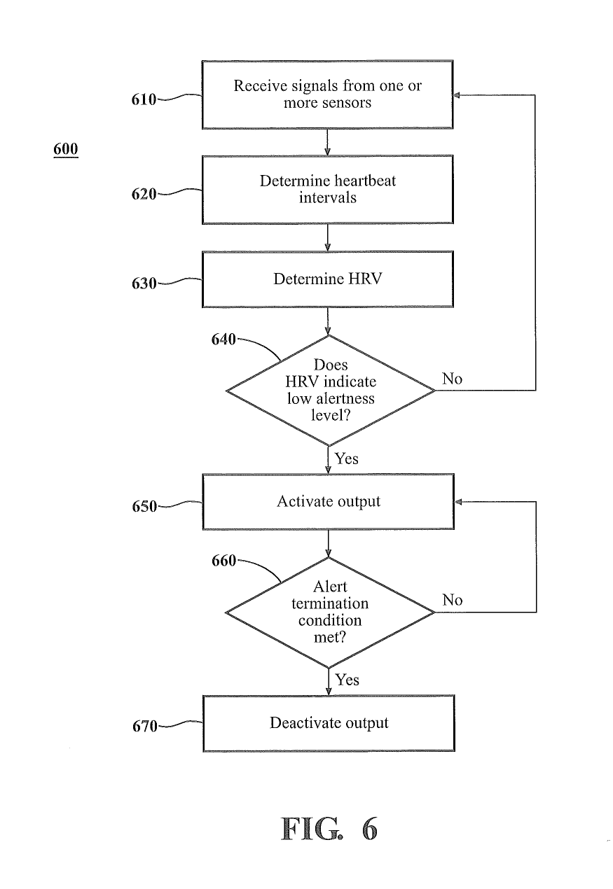

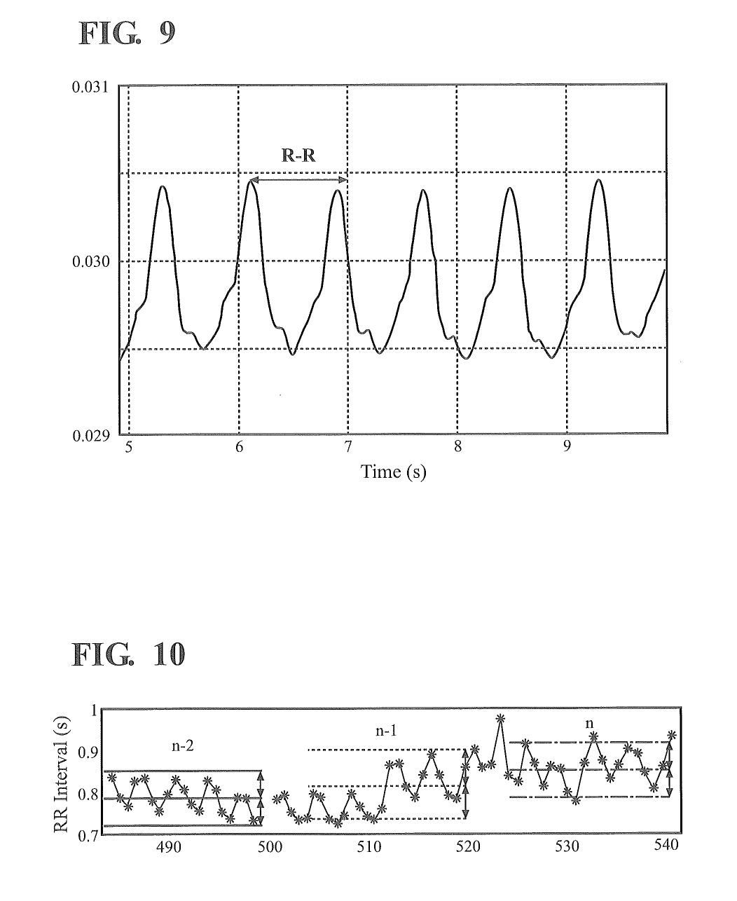

Methods of determining a low alertness level or potential drowsy state of a driver of a vehicle are described in further detail below with reference to FIGS. 6 to 14. Specifically, referring to FIG. 6, a method (600) of determining an alertness level of the driver and activating an output device is shown.

The method includes receiving signals (610) from the one or more sensors 110 which are indicative of or provide information relating to the driver's heartbeat. Each received signal is processed to determine intervals (620) of the driver's heartbeat. Depending on the type of sensor 110 and signal received, a variation in an electric, magnetic, or mechanical property of the sensor signal over time provides information from which the intervals of the driver's heartbeat can be determined. In some embodiments, this determination includes a preliminary action to filter the received signals in order to remove noise and improve or clean up the waveform for further analysis.

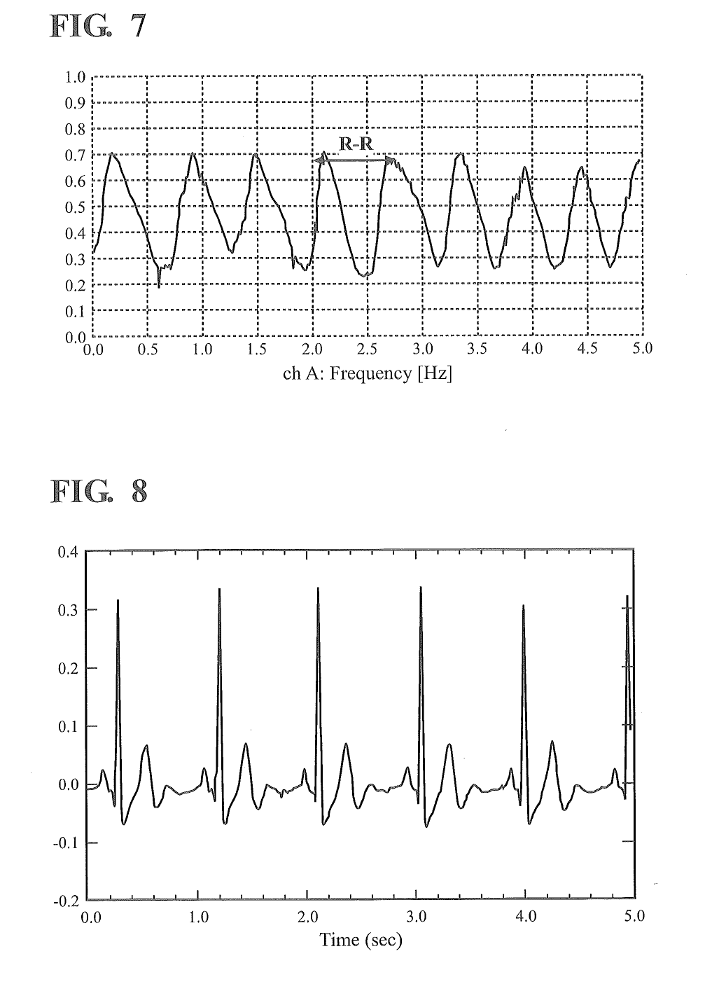

Although the measured property and amplitude of each heartbeat signal may vary from sensor to sensor, each signal may be analyzed to detect peaks in the waveforms. The three central deflections of a heartbeat waveform which are easiest to see and detect are referred to as the QRS complex. The R wave component of the QRS complex has the largest positive amplitude and HRV may be determined based on measured intervals in the peak of the R wave. The space between peaks may be referred to as the “R-R” interval and an R-R interval may be determined for each received signal and/or for the group of signals from multiple sensors. FIGS. 7, 8 and 9 illustrate sample waveforms and R-R intervals for signals received from a variety of sensor types. The present application describes the determination of HRV based on R-R intervals but other peaks or points in the heartbeat waveform may be used to determine the interval between heartbeats.

Once R-R intervals are determined for each of the signals from one or more sensors 110, the HRV is determined (630). The action of determining the HRV may include determining R-R interval outliers and omitting the outliers from the sampled data. In one embodiment, only R-R intervals inside the range 0.26 seconds <R-R <1.2 seconds are counted. This range of R-R intervals is associated with a heart rate between 50 to 230 bpm. In one embodiment, normalized data points will be assumed as the average of the previous data points.

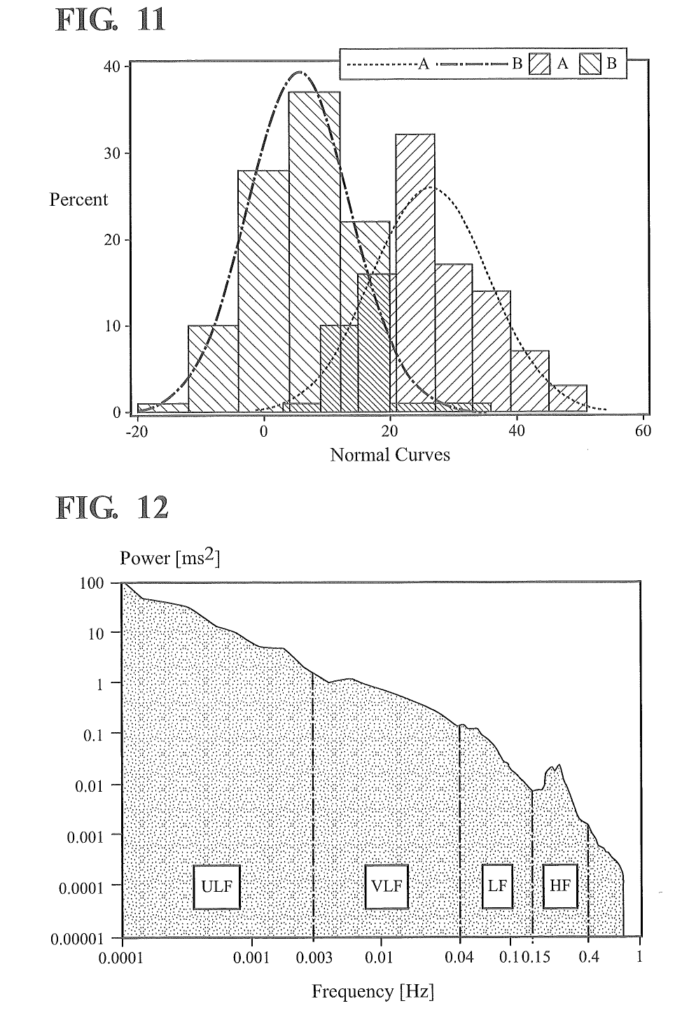

The HRV may be determined in two ways. According to the first method, R-R intervals are plotted and HRV is determined (630) based on how scattered the data points are from an average within specific clusters of time. The data points will be clustered within specific time periods and compared with the previous time period to determine the degree of variability from time period “n” to time period “n−1”, “n−2”, etc. as illustrated in FIG. 10. The data also may be normalized and analyzed based on overlapping histograms for different time periods, identified as time periods A and B in FIG. 11. The variance and the mean change may be used to compare different time periods and determine changes in HRV. Since the data is collected and processed in real time, any shift within a sub-group of a given sample size may be monitored to anticipate a trend in the data points. In other words, a smaller time period inside the broader time period, such as a group of data points within time period n, also may be analyzed to predict trends in HRV and a corresponding alertness level of the driver.

A second method to determine HRV (630) is to process the received data through power spectral analysis. Any sinusoidal or wave form signal with an amplitude that varies over time has a corresponding frequency spectrum. In one embodiment, finite time periods can be used to sample the data received from a sensor 110, such as over 3, 5, 10 or 15 minute periods. From the raw data obtained by the sensor 110, a polynomial curve fitting is done to determine a base function over time. A Fourier Series is used to describe the function over time, according to the standard expression shown in equation (1) with coefficients a₀, aₙ and bₙ as shown in equations (2), (3) and (4).

$$\begin{matrix}f ( x ) = \frac{1}{2} {a}_{0} + {\sum }_{n = 1}^{∞} ( {a}_{n} cos ( nx ) ) {\sum }_{n = 1}^{∞} ( {b}_{n} sin ( nx ) ) & ( 1 ) \\ {a}_{0} = \frac{1}{2} {\int }_{- π}^{+ π} f ( x ) {d}_{x} & ( 2 ) \\ {a}_{n} = \frac{1}{π} {\int }_{- π}^{+ π} f ( x ) cos nx & ( 3 ) \\ {b}_{n} = \frac{1}{n} {\int }_{- π}^{+ π} f ( x ) sin nx {d}_{x} & ( 4 )\end{matrix}$$

An approximation of the Fast Fourier Transform (FFT) may be achieved by computing the Discrete Fourier Transform (DFT) and converting the function from a time to a frequency domain as shown in equations (5) and (6).

F(k)=∫_{-∞}^{+∞}f(x)e^{-πikx}dₓ (5)

DFT=Xₛ=Σⱼ₌₀^{N-1}(xⱼe^{-2πijs/N}) (6)

The function f(x) is obtained by data point interpolation using the Fourier series to describe its sinusoidal behavior. The function f(x) describes the variation in the signal from the sensor 110 in regards to its independent variable. In the above equations, the independent variable x is time, and f(x) is the signal variation in milliVolts (mV) over time.

F(k) is the Fourier Transform for the infinite number of data points the sensor 110 can collect over time. Since the time period evaluation will be finite, to simplify the computational analysis, the DFT may be used for a given time period. In that case, N is the number of outputs (R-R intervals for example) and s is the continuous variable x (time) which may be replaced by a discrete variable s (an integer of “x”—which is to be confirmed by the operational data).

FIG. 12 represents a typical power spectra determined for a received signal from a sensor 110 according to equation (7). The power spectrum is achieved by squaring the modulus of F(k).

|F(k)|²=|∫_{-∞}^{+∞}f(x)e^{-2πikx}dₓ|² (7)

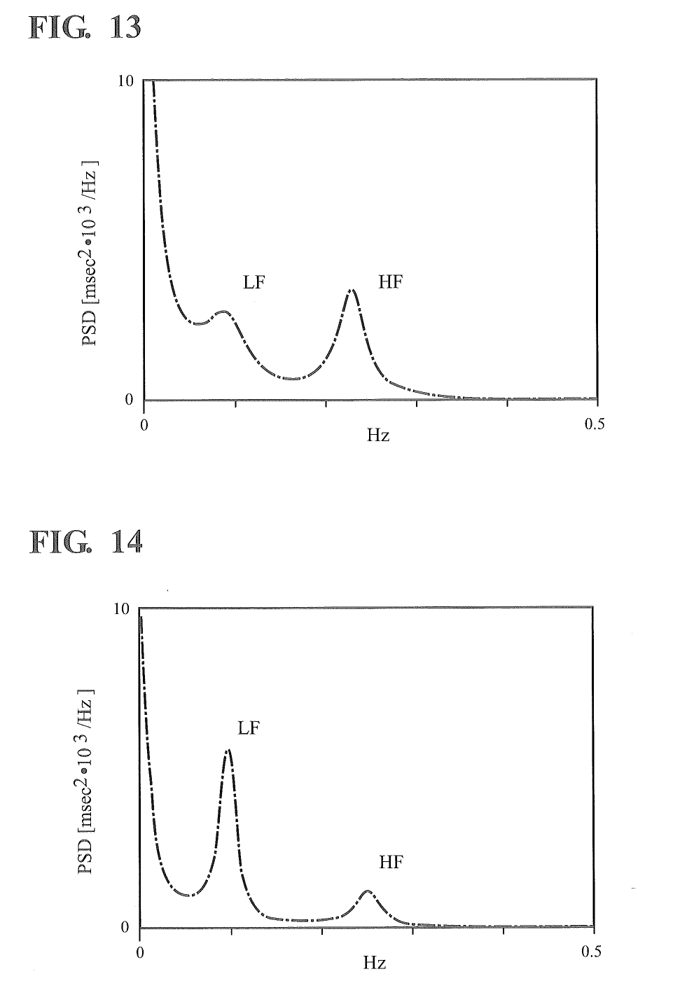

As seen in FIG. 12, data is divided into four frequency bands, ultra-low frequency (ULF), very low frequency (VLF), low frequency (LF) and high frequency (HF). FIGS. 13 and 14 illustrate a comparison between two different discrete time periods to show the fluctuations in frequency bands. The fluctuations are associated with transitional stages from alertness (FIG. 13) to drowsiness (FIG. 14), especially in the high frequency and low frequency bands.

As HRV levels decrease, a state of drowsiness or low alertness level is predicted (640). A state of drowsiness or low alertness may be predicted, for example, in response to the HRV level falling below a first predetermined threshold and/or remaining below the first predetermined threshold for a first period of time. In response to this prediction, an output device 130 is activated (650) in order to send a notification to the driver of the vehicle. Audio, visual, audio/visual or sensory notifications may be generated by one or more output devices 130 as described above. If a state of drowsiness or low alertness is not predicted, the method (600) continues to receive signals from the sensors 110, determine heartbeat intervals (620), and determine and monitor HRV levels (630).

If a termination condition (660)is met, the one or more output devices 130 may be deactivated (670) in order to stop generating warnings or alarms. The termination condition (660) may include the HRV level increasing above a second predetermined threshold and/or remaining above the second predetermined threshold for a second period of time. The first and second predetermined thresholds may or may not be the same. The first and second periods of time may or may not be the same. As described above, a termination condition (660) may include the receipt of an input through a user interface of the system 100 or through an input of the infotainment system, to acknowledge the alarm or warning signal, or the slowing or stopping of the vehicle, or a combination of these inputs and conditions along with the measured HRV level.

Although the exemplary embodiments described herein employ device memory, other types of computer readable media which can store data that are accessible by a computer, such as magnetic cassettes, flash memory cards, digital versatile disks, cartridges, random access memories (RAMs), read only memory (ROM), USB or memory sticks, a cable or wireless signal containing a bit stream and the like, also may be used in the exemplary operating environment. Non-transitory computer-readable storage media expressly exclude media such as energy, carrier signals, electromagnetic waves and signals per se.

The invention has been described in an illustrative manner, and it is to be understood that the terminology, which has been used, is intended to be in the nature of words of description rather than of limitation. Many modifications and variations of the present invention are possible in light of the above teachings. It is, therefore, to be understood that within the scope of the appended claims, the invention may be practiced other than as specifically described.

1. A method of detecting alertness of a driver of a vehicle comprising:
receiving, from at least one sensor in a seat assembly of the vehicle, signals indicative of a heartbeat of the driver;
determining heartbeat intervals based on the received signals;
determining heart rate variability (HRV) levels based on the heartbeat intervals; and
in response to the determined HRV levels indicating a low state of alertness of the driver, activating at least one output device.

2. The method of claim 1 wherein determining heartbeat intervals comprises determining an R-R interval between R waves of the heartbeat.

3. The method of claim 1 wherein determining HRV levels comprises determining power spectra for HRV levels over multiple time periods and comparing the power spectra to detect the low state of alertness of the driver.

4. The method of claim 1 wherein the at least one sensor comprises a capacitive sensor, a radio frequency (RF) sensor, an impedance sensor, a permittivity sensor, or a ultrasensitive pressure transducer.

5. The method of claim 1 wherein the at least one activated output device generates an audio, visual, audio/visual or haptic signal to alert the driver.

6. The method of claim 1 wherein the at least one activated output device comprises an infotainment system of the vehicle, or a device to vibrate the seat assembly, a steering wheel, or a restraint system.

7. The method of claim 1 further comprising deactivating the at least one output device in response to the determined HRV level indicating a higher state of alertness of the driver.

8. A system for detecting alertness of a driver of a vehicle comprising:
a seat assembly having at least one sensor;
at least one output device; and
a computing device configured to:
receive from the at least one sensor signals indicative of a heartbeat of the driver;
determine heartbeat intervals based on the received signals;
determine heart rate variability (HRV) levels based on the heartbeat intervals; and
in response to the determined HRV levels indicating a low state of alertness of the driver, activate the at least one output device.

9. The system of claim 8 wherein the at least one sensor comprises a capacitive sensor, a radio frequency (RF) sensor, an impedance sensor, a permittivity sensor, or a ultrasensitive pressure transducer.

10. The system of claim 8 wherein the at least one sensor is mounted behind a trim cover assembly of the seat assembly.

11. The system of claim 8 wherein the at least one sensor is integrated into the trim cover assembly of the seat assembly.

12. The system of claim 8 wherein the at least one output device comprises an infotainment system of the vehicle, or a device to vibrate the seat assembly, a steering wheel, or a restraint system.

13. The system of claim 8 further comprising an electrostatic discharge mat located in a seat cushion of the seat assembly and grounded to the vehicle.

14. The system of claim 8 wherein the computing device is further configured to deactivate the at least one output device in response to the determined HRV level indicating a higher state of alertness of the driver.

15. The system of claim 8 wherein the computing device comprises a processor and a memory.
